(19) United States
(12) Patent Application Publication
(10) Pub. No.: US 2012/0296155 A1
(43) Pub. Date: Nov. 22, 2012

(54) Magnetic Attachment Arrangement for Implantable Device

(75) Inventor: Geoffrey R. Ball, Axams (AT)

(73) Assignee: VIBRANT MED-EL HEARING TECHNOLOGY GMBH, Innsbruck (AT)

(21) Appl. No.: 13/550,730

(22) Filed: Jul. 17, 2012

Related U.S. Application Data
(63) Continuation-in-part of application No. 13/462,931, filed on May 3, 2012.
Application No. 13/462,931 is a division of application No. 12/839,887, filed on Jul. 20, 2010, now abandoned.
Application No. 12/839,887 is a continuation-in-part of application No. 13/091,352, filed on Apr. 21, 2011.
(60) Provisional application No. 61/227,632, filed on Jul. 22, 2009.
(60) Provisional application No. 61/327,158, filed on Apr. 23, 2010.

(51) Int. Cl. A61F 11/00; H04R 25/00

(52) U.S. Cl. 600/25; 607/57

(57) ABSTRACT

An arrangement is described for an implantable medical system. An implant housing contains a portion of an implantable electronic system and has a planar outer surface adapted to lie parallel to overlying skin in an implanted patient. An implant magnet arrangement is located within the housing and adapted to magnetically interact with a corresponding external magnet in an external device on the skin of the implanted patient over the implant housing. The implant magnet arrangement includes an inner center disc having a magnetic dipole parallel to the planar outer surface of the implant housing with an inner magnetic orientation in an inner magnetic direction, and an outer radial ring having a magnetic dipole parallel to the planar outer surface of the implant housing with an outer magnetic orientation in an outer magnetic direction opposite to the inner magnetic direction.

This application is a continuation in part of U.S. patent application Ser. No. 13/462,931, filed May 3, 2012, which is a divisional of U.S. patent application Ser. No. 12/839,887, file Jul. 20, 2010, which in turn claimed priority from U.S. Provisional Patent Application 61/227,632, filed Jul. 22, 2009; and this application also is a continuation in part of U.S. patent application Ser. No. 13/091,352, filed Apr. 21, 2011, which in turn claims priority from U.S. Provisional Patent Application 61/327,158, filed Apr. 23, 2010; all of which are incorporated herein by reference.

### FIELD OF THE INVENTION

The present invention relates to medical implants, and more specifically to a permanent magnet arrangement for use in such implants.

### BACKGROUND ART

Some hearing implants such as Middle Ear Implants (MEI's) and Cochlear Implants (CI's) employ attachment magnets in the implantable part and an external part to hold the external part magnetically in place over the implant. For example, as shown in FIG. 1, a typical cochlear implant system may include an external transmitter housing 101 containing transmitting coils 102 and an external magnet 103. The external magnet 103 has a conventional coin-shape and a north-south magnetic dipole that is perpendicular to the skin of the patient to produce external magnetic field lines 104 as shown. Implanted under the patient's skin is a corresponding receiver assembly 105 having similar receiving coils 106 and an implanted internal magnet 107. The internal magnet 107 also has a coin-shape and a north-south magnetic dipole that is perpendicular to the skin of the patient to produce internal magnetic field lines 108 as shown. The internal receiver housing 105 is surgically implanted and fixed in place within the patient's body. The external transmitter housing 101 is placed in proper position over the skin covering the internal receiver assembly 105 and held in place by interaction between the internal magnetic field lines 108 and the external magnetic field lines 104. Rf signals from the transmitter coils 102 couple data and/or power to the receiving coil 106 which is in communication with an implanted processor module (not shown).

One problem arises when the patient undergoes Magnetic Resonance Imaging (MRI) examination. Interactions occur between the implant magnet and the applied external magnetic field for the MRI. As shown in FIG. 2, the direction magnetization m̅ of the implant magnet 202 is essentially perpendicular to the skin of the patient. Thus, the external magnetic field B̅ from the MRI may create a torque T̅ on the internal magnet 202, which may displace the internal magnet 202 or the whole implant housing 201 out of proper position. Among other things, this may damage the adjacent tissue in the patient. In addition, the external magnetic field B̅ from the MRI may reduce or remove the magnetization m̅ of the implant magnet 202 so that it may no longer be strong enough to hold the external transmitter housing in proper position. The implant magnet 202 may also cause imaging artifacts in the MRI image, there may be induced voltages in the receiving coil, and hearing artifacts due to the interaction of the external magnetic field B̅ of the MRI with the implanted device. This is especially an issue with MRI field strengths exceeding 1.5 Tesla.

Thus, for existing implant systems with magnet arrangements, it is common to either not permit MRI or at most limit use of MRI to lower field strengths. Other existing solutions include use of a surgically removable magnets, spherical implant magnets (e.g. U.S. Pat. No. 7,566,296), and various ring magnet designs (e.g., U.S. Provisional Patent 61/227,632, filed Jul. 22, 2009). Among those solutions that do not require surgery to remove the magnet, the spherical magnet design may be the most convenient and safest option for MRI removal even at very high field strengths. But the spherical magnet arrangement requires a relatively large magnet much larger than the thickness of the other components of the implant, thereby increasing the volume occupied by the implant. This in turn can create its own problems. For example, some systems, such as cochlear implants, are implanted between the skin and underlying bone. The “spherical bump” of the magnet housing therefore requires preparing a recess into the underlying bone. This is an additional step during implantation in such applications which can be very challenging or even impossible in case of very young children.

Various complicated arrangements of magnetic elements have been described for use in therapeutic applications; see for example, U.S. Pat. No. 4,549,532 and U.S. Pat. No. 7,608,035. However, there is no suggestion that such therapeutic arrangements might potentially have any utility for magnetic attachment applications such as those described above.

### SUMMARY OF THE INVENTION

Embodiments of the present invention are directed to an arrangement for an implantable medical system. An implant housing contains a portion of an implantable electronic system and has a planar outer surface adapted to lie parallel to overlying skin in an implanted patient. An implant magnet arrangement is located within the housing and adapted to magnetically interact with a corresponding external magnet in an external device on the skin of the implanted patient over the implant housing. The implant magnet arrangement includes an inner center disc having a magnetic dipole parallel to the planar outer surface of the implant housing with an inner magnetic orientation in an inner magnetic direction, and an outer radial ring having a magnetic dipole parallel to the planar outer surface of the implant housing with an outer magnetic orientation in an outer magnetic direction opposite to the inner magnetic direction.

There also may be an implant signal coil within the implant housing which surrounds the implant magnet arrangement for transcutaneously receiving an externally generated communication signal. The implant housing may be made of titanium. The implant magnet arrangement may be hermetically encapsulated within the implant housing. There may also be a similar external housing having a corresponding magnet arrangement. The implantable electronic system may be, for example, a vestibular implant system, a cochlear implant system, a middle ear implant system, or a bone conduction hearing implant system.

### BRIEF DESCRIPTION OF THE DRAWINGS

FIG. 1 shows a portion of a typical idealized cochlear implant which may be used in embodiments of the present invention.

FIG. 2 shows effects of an external magnetic field on an implanted portion of an implanted device which may be used in embodiments of the present invention.

FIG. 3A-B shows an implant magnet arrangement according to embodiments of the present invention.

FIG. 4 shows how an embodiment of an implant magnet arrangement cooperates with a typical external device.

FIG. 5 shows how an embodiment of an implant magnet arrangement cooperates with another corresponding external magnet arrangement.

FIG. 6A shows the magnetic field arrangement in typical existing implant attachment magnets.

FIG. 6B shows an embodiment of an implant magnet arrangement having a magnetic dipole oriented across the diameter of the attachment magnet parallel to the plane of the coil housing.

FIG. 7A-B shows an elevated perspective view and a side cross-sectional view respectively of a portion of a cochlear implant system having an implant magnet arrangement with a magnetic dipole parallel to the plane of the coil housing.

FIG. 8A-B shows an elevated perspective view and a side cross-sectional view respectively of an implant magnet arrangement according to another embodiment of the present invention having an inner disk magnet with a magnetic dipole parallel to the plane of the coil housing in a first direction and an outer ring magnet with a magnetic dipole parallel to the plane of the coil housing in an opposite second direction.

FIG. 9 shows a side cross-sectional view of an implant and external magnets similar to the embodiment in FIG. 8.

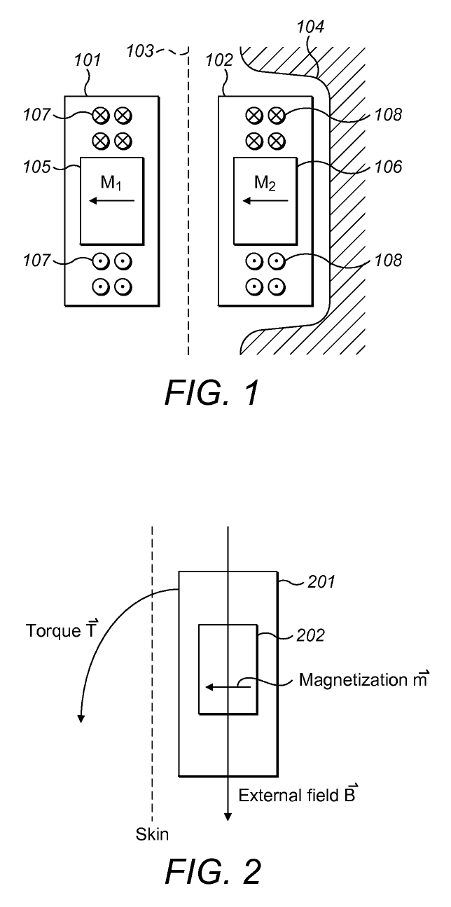

### DETAILED DESCRIPTION OF SPECIFIC EMBODIMENTS

Various embodiments of the present invention are directed to an improved magnet arrangement for implantable devices in the form of a cylindrical magnet having multiple adjacent magnetic sections wherein at least two of the magnetic sections have opposing magnetic orientations in opposite magnetic directions.

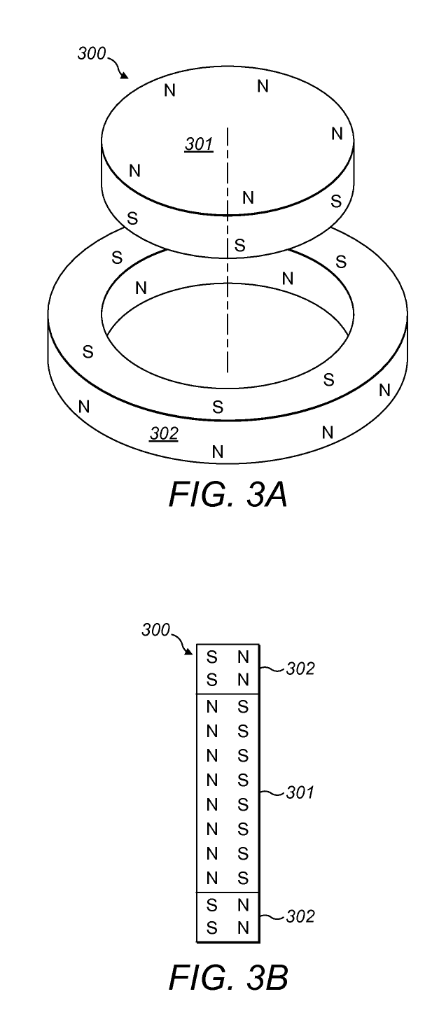

FIG. 3A shows an exploded elevated view and FIG. 3B shows a side view of an implant magnet arrangement 300 according to embodiments of the present invention. An implantable housing (e.g., implant housing 102) contains a portion of an implantable electronic system. The implantable electronic system may be, for example, a vestibular implant system, a cochlear implant system, a middle ear implant system, or a bone conduction hearing implant system. A cylindrical implant magnet arrangement 300 within the housing includes an inner center disc section 301 having an inner magnetic orientation in an inner magnetic direction, and an outer radial ring section 302 having an outer magnetic orientation in an outer magnetic direction opposite to the inner magnetic direction.

With such an arrangement, the net magnetic field of the implant magnet arrangement 300 is much less than in the conventional cylindrical magnet of the prior art, while locally the magnetic fields are still effectively strong near the inner center disc section 301 and the outer radial ring section 302 so that there is no overall loss in the retention force of the implant magnet arrangement 300. Such a reduced net magnetic field of the implant magnet arrangement 300 also avoids the prior problems of the net magnetic fields adversely interacting with the implant signal coil and its communications signal and reduces the torque and imaging problems of the prior art with regards to MRI procedures. Moreover, the greater specificity of the magnetic structures of the implant magnet arrangement 300 compared with a simple disk magnet also provides improved centering capability with regards to the external component housing.

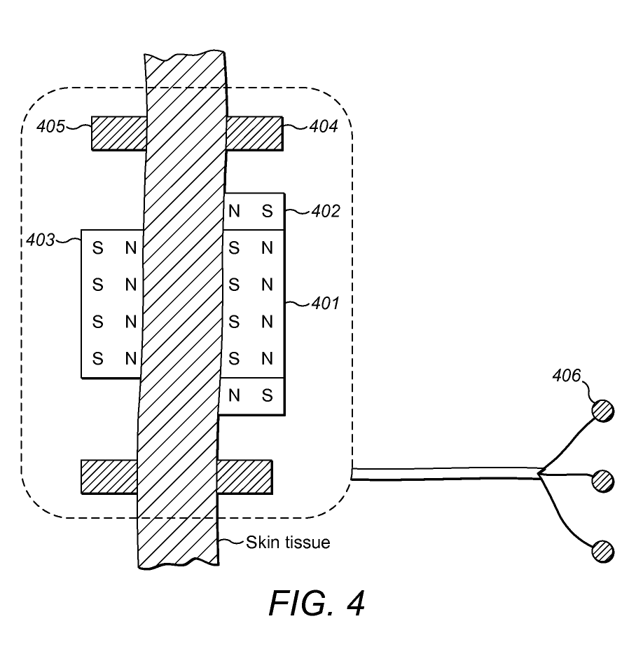

FIG. 4 shows how an embodiment of an implant magnet arrangement cooperates with a typical external device. A conventional cylindrical external magnet 403 interacts with an implant magnet having an inner center disc section 401 and an outer radial ring section 402 according to an embodiment of the invention. In this case, the external magnet 403 is similar in diameter to the inner center disc section 401 of the implant magnet so that their respective magnetic fields interact to provide the desired retention force to hold the external device in proper operating position. This allows external signal coil 405 to couple an implant communications signal containing data and power through to a corresponding implant coil 404. The implant communications signal received by the implant coil 404 then is coupled to other elements 406 of the implant system such as an implant processor of a cochlear implant, bone conduction transducer, or middle ear transducer. In some embodiments, there may be multiple implant magnet arrangements and corresponding external magnets.

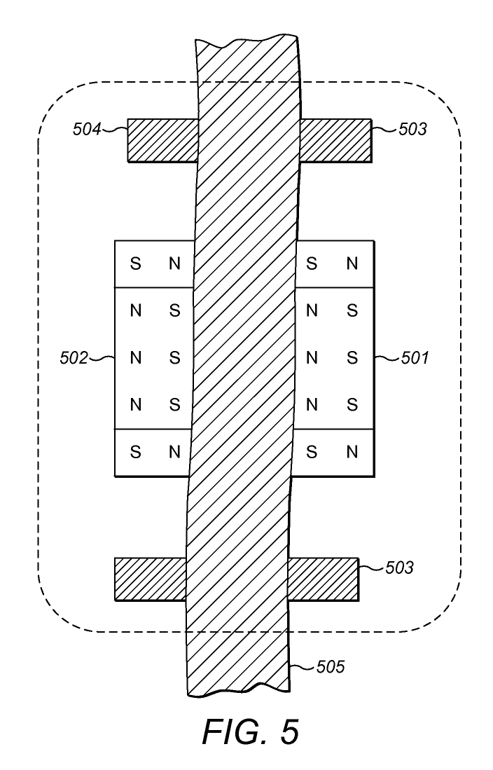

FIG. 5 shows how an embodiment of an implant magnet arrangement cooperates with another corresponding external magnet arrangement. In this case, the external magnet 502 also has inner and outer sections that correspond to similar sections of the implant magnet 501 to cooperate to hold the external device in proper operating position. In some embodiments, there may be multiple implant magnet arrangements. This allows an external signal coil 504 to couple an implant communications signal containing data and power across the skin 505 to a corresponding implant coil 503 for use by other elements of the implant system.

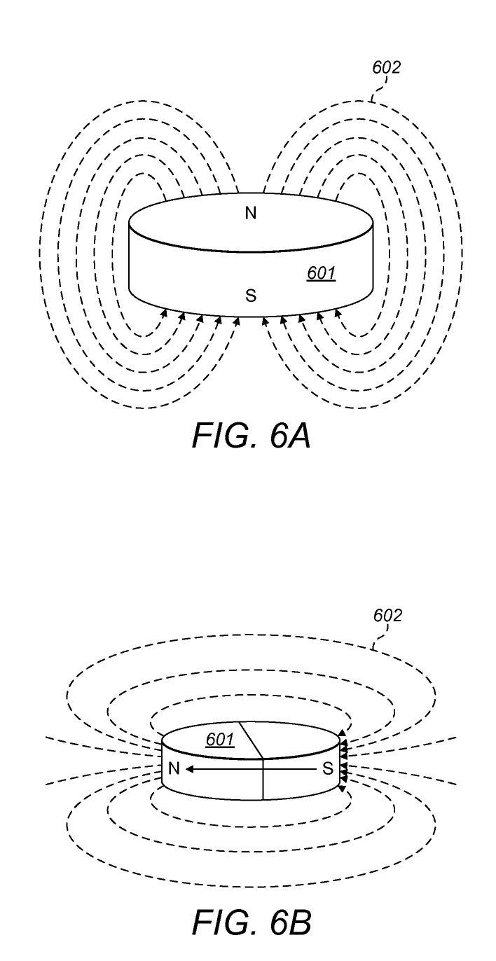

FIG. 6A shows the magnetic field arrangement in typical existing implant attachment magnets. In this case, the attachment magnet 601 is disk-shaped (i.e., cylindrical) with the north-south magnetic dipole realized in the axial direction as is conventional producing magnetic field lines 602 as shown. The magnetic arrangement shown in FIG. 6B changes the direction of magnetization so that the north-south magnetic dipole is oriented across the diameter of the attachment magnet 601 parallel to (i.e., “in”) the plane of the coil housing, producing magnetic field lines 602 as shown.

Of course, with such an arrangement, it is important that both the internal implant receiver attachment magnet and the external transmitter attachment magnet be magnetized with the same orientation in the plane of the coil housing (i.e., parallel to the skin). Then when the external coil housing is placed onto the patient's skin over the implant coil housing, the two attachment magnets turns around on their axis such that the north and south poles of one attachment magnet are positioned adjacent to south and north poles respectively of the other attachment magnet thereby maximizing the attractive magnetic force between the two.

With such an arrangement, the net magnetic field of the implant magnet arrangement 600 is much less than in the conventional cylindrical magnet of the prior art, while locally the magnetic fields are still effectively strong near the inner center disc section 601 and the outer radial ring section 602 so that there is no overall loss in the retention force of the implant magnet arrangement 600. Such a reduced net magnetic field of the implant magnet arrangement 600 also avoids the prior problems of the net magnetic fields adversely interacting with the implant signal coil and its communications signal and reduces the torque and imaging problems of the prior art with regards to MRI procedures. Moreover, the greater specificity of the magnetic structures of the implant magnet arrangement 600 compared with a simple disk magnet also provides improved centering capability with regards to the external component housing.

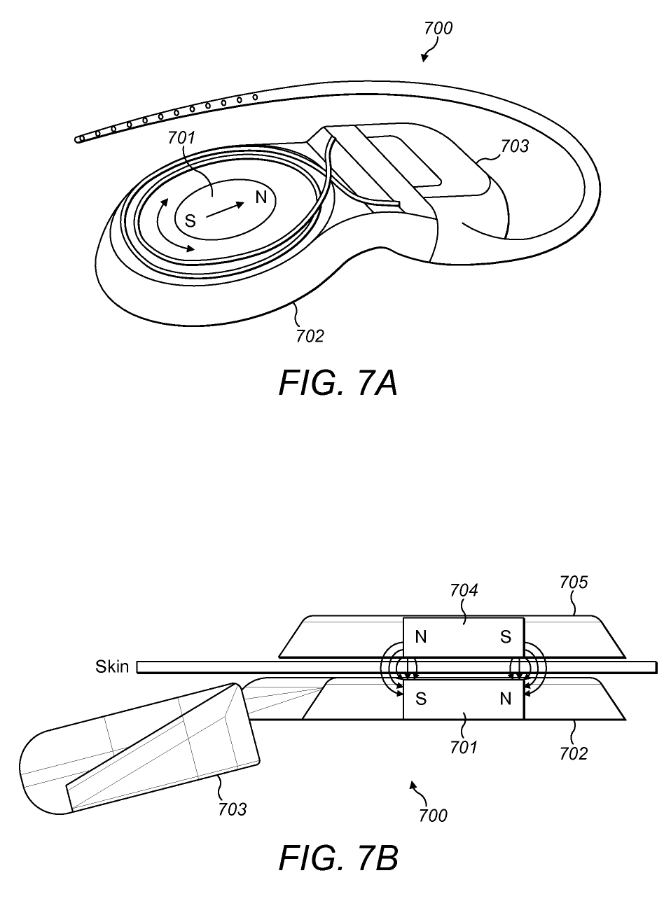

FIG. 7A shows an elevated perspective view and FIG. 7B shows a side cross-sectional view of a cochlear implant 700 having a planar coil housing 702 that contains a signal coil for transcutaneous communication of an implant communication signal. A first attachment magnet 701 is located within the plane of the coil housing 702 and rotatable therein (e.g., a planar disk shape) has a magnetization direction with a magnetic dipole parallel to the plane of the coil housing 702. An external transmitter coil housing 705 with a corresponding second attachment magnet 704 with a similar magnetic dipole direction parallel to the plane of its coil housing 705 so that when placed on the skin of the recipient patient, their respective magnetic fields cause the two attachment magnets 701 and 704 to self-orient as described above to form a magnetic attraction connection between them. In specific embodiments, the coil housing 702 may be made have a titanium case with the attachment magnet 701 located outside the titanium case, for example, embedded in a silicone coil assembly. Alternatively, the coil housing 702 may be a ceramic case where the attachment magnet 701 is hermetically encapsulated within the ceramic housing.

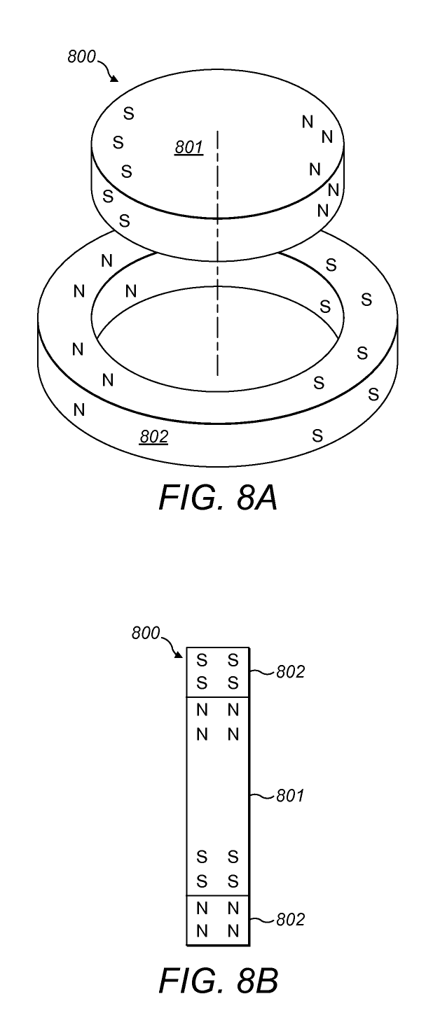

FIG. 8A-B shows an elevated perspective view and a side cross-sectional view respectively of an implant magnet arrangement 800 according to another embodiment of the present invention. An inner disk magnet 801 has a magnetic dipole across its diameter parallel to the plane of the coil housing in a first inner magnetic direction. An outer ring magnet 802 has a magnetic dipole across its diameter parallel to the plane of the coil housing in a second outer magnetic direction which is opposite to the inner magnetic direction.

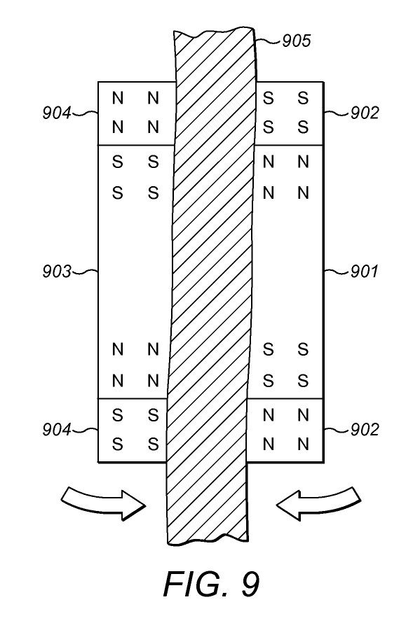

FIG. 9 shows a side cross-sectional view of implant magnets 901 and 902 and external magnets 903 and 904 which are similar to the embodiment in FIG. 8, and magnetically interact with each other across the skin 905 of the implanted patient. The implant magnets may typically be hermetically encapsulated within a planar coil housing (not shown) made of titanium. The coil housing also typically contains a signal coil for transcutaneous receiving an externally generated implant communication signal, and a portion of an implantable electronic system, which may be, for example, a vestibular implant system, a cochlear implant system, a middle ear implant system, or a bone conduction hearing implant system. The external magnets 903 and 904 are located within an external device (not shown) and have a magnetic field arrangement and orientation which is similar to but opposite to that of the implant magnets 901 and 902 so as to magnetically interact with them to hold the external device in proper operating position.

Implant magnets according to embodiments of the present invention present a slim profile which is safe for MRI field strengths up to and beyond 3 Tesla without the need to surgically remove the implant magnet. Alternatively, in some embodiments the implant attachment magnet may be adapted to be temporarily removable by minor surgery from the implant coil housing if desired to reduce MRI artifacts.

In contrast to spherical design attachment magnets, the present coil housing can have a flat bottom so that there is no need to drill a recess into the bone during implantation of the device. This makes such a magnet design especially well-suited for implantation in young children. Moreover, embodiments can be equally effective where there is a relatively large magnet in the implanted part and a relatively small magnet in the external part, and vice versa. And due to the different magnetization direction, it is expected that the MR imaging artifact may be smaller compared to conventional implant magnets, for example, extending less in the medial direction.

Compared to the conventional disk magnet concept with axial magnetization, embodiments of the present invention have attractive forces on both poles, and the attraction is caused by two forces which apply at the two poles of each magnet. The result is that the shear force between the external attachment magnet and the implant attachment magnet is higher in the direction of the magnetization axis of the two magnets. By turning the external attachment magnet for optimal orientation over the implant (e.g. vertical magnetic axis), a better magnetic attachment of the external parts can be achieved. In such an arrangement, the external attachment magnet also stays in place over the implant attachment magnet with less lateral displacement even in response to small mechanical shocks. The present embodiments also have a better (shallower) force-over-distance diagram than two conventional magnets with axial magnetization. It may be advantageous if the attractive force does not vary greatly over the distance between the two attachment magnets.

With standard supine patient position where the implant attachment magnet is oriented in a coronal plane, embodiments of the attachment magnet described here can align well with the static magnetic field in closed MR scanners only while such an implant magnet in axial orientation would only align with the static magnetic field in open scanners with vertical magnetic field. The torque exerted to the implant can remain relatively high when the implant magnet which has only one degree of freedom cannot align well enough with the external magnetic field.

Embodiments of the present invention such as those described above can be easily and directly implemented in existing products with corresponding size and geometry replacement magnets, either for the implanted magnet and/or the external magnet. Embodiments may usefully contain permanent magnetic material and/or ferro-magnetic material as well as other structural materials. These include without limitation magnetic ferrite materials such as Fe₃O₄, BaFe₁₂O₁₉ etc., compound materials such as plastic bonded permanent magnetic powder, and/or sintered material such as sintered NdFeB, SmCo, etc. Selection of the proper materials and arrangements may help avoid or reduce undesired eddy currents.

Although various exemplary embodiments of the invention have been disclosed, it should be apparent to those skilled in the art that various changes and modifications can be made which will achieve some of the advantages of the invention without departing from the true scope of the invention.

What is claimed is:

1. An arrangement for an implantable medical system comprising:
an implant housing containing a portion of an implantable electronic system and including a planar outer surface adapted to lie parallel to overlying skin in an implanted patient; and
an implant magnet arrangement within the housing adapted to magnetically interact with a corresponding external magnet in an external device on the skin of the implanted patient over the implant housing, the implant magnet arrangement including:
i. an inner center disc having a magnetic dipole parallel to the planar outer surface of the implant housing with an inner magnetic orientation in an inner magnetic direction, and
ii. an outer radial ring having a magnetic dipole parallel to the planar outer surface of the implant housing with an outer magnetic orientation in an outer magnetic direction opposite to the inner magnetic direction.

2. An arrangement according to claim 1, further comprising:
an implant signal coil within the implant housing, surrounding the implant magnet arrangement for transcutaneously receiving an externally generated communication signal.

3. An arrangement according to claim 1, further comprising:
an external device containing the external magnet and adapted for magnetic attachment on the skin of the implanted patient.

4. An arrangement according to claim 1, wherein the implant housing is made of titanium.

5. An arrangement according to claim 1, wherein the implant magnet arrangement is hermetically encapsulated within the implant housing.

6. An arrangement according to claim 1, wherein the implantable electronic system includes a middle ear implant system.

7. An implantable device according to claim 1, wherein the implantable electronic system includes a bone conduction hearing implant system.

8. An arrangement according to claim 1, wherein the implantable electronic system includes a vestibular implant system.
